(19) United States
(12) Patent Application Publication
(10) Pub. No.: US 2004/0086747 A1
(43) Pub. Date: May 6, 2004

(54) Azepine compounds

(76) Inventor: Masaki Matsui, Seki-shi (JP)

Correspondence Address: BIRCH STEWART KOLASCH & BIRCH, PO BOX 747, FALLS CHURCH, VA, 22040-0747, US

(21) Appl. No.: 10/673,449

(22) Filed: Sep. 30, 2003

(30) Foreign Application Priority Data
Oct. 1, 2002 (JP) 289039/2002

(51) Int. Cl. H05B 33/14; C07D 243/08

(52) U.S. Cl. 428/690; 428/917; 313/504; 313/506; 540/553; 540/575

(57) ABSTRACT

A light-emitting layer of an organic electroluminescent device comprises the following compound (I):

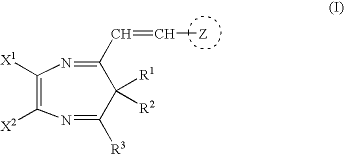

wherein X¹ and X² are a cyano group; R¹ is a C₁-₆alkyl group; R² is a hydrogen atom or a C₁₋₆alkyl group; R³ is a hydrogen atom or a C₁₋₆alkyl group; the ring Z represents an aromatic hydrocarbon ring which has a substituent such as a N-substituted amino group, or a heterocycle which has a substituent such as a N-substituted amino group. The present invention provides an azepine compound useful for a light emission material of organic electroluminescent devices, and a process for producing the same.

### FIELD OF THE INVENTION

The present invention relates to novel azepine compounds useful for functional materials (particularly, materials applicable to organic electroluminescent devices), a process for producing the same, and an organic electroluminescent device using the same.

### BACKGROUND OF THE INVENTION

As a fluorescent pigment, a compound having a planar structure and a hard or rigid π-conjugated system (e.g., stilbene, coumarin, naphthalimide, perylene, Rhodamine) has been known. Meanwhile, a fluorescent pigment such as a pyrazine ring-containing compound (e.g., styryl pyrazine, 2,5-bis(dialkylamino)-3,6-dicyanopyrazine, a pyrazino heterocyclic compound, pyrazino phthalocyanine) has been also known. Since these pigments not only emit fluorescent light upon light irradiation but have such functions as light absorption (e.g., color, pleochroism), photoconductivity, and reversible changes by heat or light (e.g., thermochromism, photochromism), these pigments have been used as functional materials in a variety of fields (e.g., fluorescent materials, photochromic materials, optical recording materials). In particular, those that emit light by the action (application) of electric fields are useful for emission center compounds in organic electroluminescent devices (hereinafter, may refer to as organic EL devices) which are desired to be fully colored.

In these organic EL devices, colors emitted by organic EL devices can be selected by suitably selecting an emission center compound for the light-emitting layer. For example, Japanese Patent Application Laid-Open No. 73443/1996 (JP-8-73443A) discloses a dimer of pyrazine in which a pyrazine group having a phenyl group is bound to a divalent aromatic group, and an organic EL device containing the pyrazine derivative in an organic layer. However, the pyrazine dimer emits blue light having a relatively shorter wavelength. The electroluminescent device is therefore restricted to its emission wavelength and hardly emits a light in the red region light in spite of requiring a fluorescent pigment capable of emitting light in the red region.

In particular, since a pigment is used in a high concentration or in the form of solid thin film in an organic electroluminescent device, a fluorescent pigment whose molecular structure has a planar backbone is easy to cause concentration quenching. Japanese Patent Application Laid-Open No. 145869/2002 (JP-2002-145869A) discloses an azepine compound as a useful compound for an organic electroluminescent device, and also describes that the azepine compound has a non-planer structure.

### SUMMARY OF THE INVENTION

It is therefore an object of the present invention to provide a novel azepine compound capable of emitting light upon light irradiation or by the action of electric fields and useful for a functional material such as a material for an organic EL device, a process for producing the same, and an organic EL device using the same.

Another object of the present invention is to provide an azepine compound which is capable of emitting light of longer wavelength (e.g., light emission in the red region) at a high emission luminance or intensity, and an organic EL device using the same.

The inventor of the present invention made intensive studies to achieve the above objects and finally found that a compound, in which a specific ring is bonded to an azepine ring having a specific substituent, via a C═C double bond, emits light upon light irradiation or by the action of electric fields and therefore is useful for a functional material of organic electroluminescent devices. The present invention was accomplished based on the above findings.

That is, the azepine compound of the present invention is represented by the following formula (I):

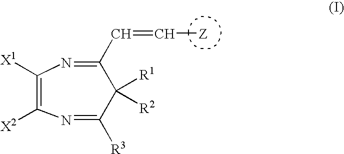

wherein X¹ and X² are the same or different, each representing an electron attractive group; R¹ and R² are the same or different, each representing a hydrogen atom, or an alkyl group, and at least one of the groups, R¹ and R², is an alkyl group; R³ represents a hydrogen atom, an alkyl group, an aryl group, an aralkyl group, or an alkoxy group; and the ring Z represents a hydrocarbon ring which may have a substituent or a heterocycle which may have a substituent.

At least one of the groups, X¹ and X², may be a cyano group. In the azepine compound, R¹ may be a C₁₋₆alkyl group, R² may be a hydrogen atom or a C₁₋₆alkyl group, and R³ may be a hydrogen atom or a C₁₋₆alkyl group. Moreover, the ring Z may be an aromatic ring, for example, a benzene ring which has an electron donative group (at least one member selected from the group consisting of an amino group, a N-substituted amino group, a hydroxyl group, an alkoxy group, a halogen atom and an alkyl group) as a substituent, on at least one of the positions, o-position and p-position. Such an azepine compound is capable of emitting light by applying a light or an electric field. The introduction of an alkyl group into at least one of the substituents, R¹ and R², in the azepine ring insures red shift (or shift to longer wavelength) of the emission wavelength.

The present invention also includes a process for producing the compound (I) which comprises reacting a compound represented by the following formula (IV) (an azepine derivative) with a compound represented by the following formula (V) (an aldehyde);

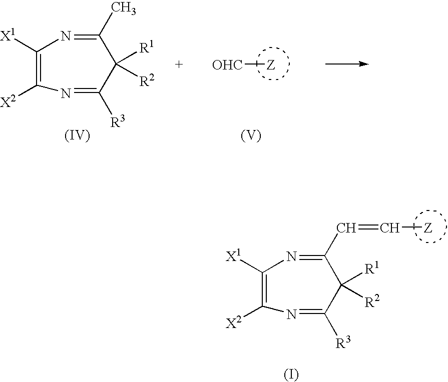

wherein X¹, X², R¹, R², R³, and the ring Z have the same meanings as defined above.

Moreover, the present invention also includes an organic electroluminescent device which comprises a pair of electrodes and an organic layer (or light-emitting layer) interposed therebetween, wherein the organic layer comprises a compound represented by the formula (I). The organic layer of the organic electroluminescent device may have (1) a single layer structure composed of a light-emitting layer having at least one function selected from the group consisting of an electron-transportability (or electron-transporting function) and a hole-transportability (or hole-transporting function), or (2) a layered structure composed of a layer having at least one function selected from the group consisting of an electron-transportability and a hole-transportability, and a light-emitting layer.

### BRIEF DESCRIPTION OF THE DRAWINGS

FIG. 1 is a graph showing the emission luminance value (cd/m²) of the organic electroluminescent devices obtained in Examples 1 and 2 and Comparative Example 1, versus voltage applied (V) thereto.

FIG. 2 is a graph showing emission spectra (luminescence intensity distribution) of the organic electroluminescent devices obtained in Examples 1 and 2 and Comparative Example 1.

### DETAILED DESCRIPTION OF THE INVENTION

In the azepine compound represented by the above formula (I), exemplified as the electron attractive group represented by X¹ and X² is a cyano group, a carbonyl group. As the electron attractive group, the cyano group is preferred. At least one of the groups, X¹ and X², is usually the cyano group, and it is preferred that both of them are cyano groups. An azepine ring having such X¹ and X² probably acts as an acceptor for intramolecular charge transfer.

The alkyl groups represented by R¹ and R² includes, for example, a linear or branched C₁₋₂₀alkyl group (e.g., a C₁₋₁₀alkyl group) such as methyl, ethyl, propyl, isopropyl, butyl, isobutyl, s-butyl, t-butyl, pentyl, hexyl, or octyl group, preferably a C₁₋₆alkyl group, and more preferably a C₁₋₄alkyl group.

At least one group of the substituents R¹ and R² is an alkyl group, and the both substituents R¹ and R² may be the same or different alkyl groups mentioned above. Regarding the substituents R¹ and R², R¹ is usually a linear or branched C₁₋₆alkyl group (e.g., a C₁₋₄alkyl group), and R² is a hydrogen atom or a linear or branched C₁₋₆alkyl group. In particular, the substituent R² is usually a hydrogen atom.

As the alkyl group represented by R³, there may be, for example, a C₁₋₂₀alkyl group (e.g., a C₁₋₁₀alkyl group) such as methyl, ethyl, propyl, isopropyl, butyl, isobutyl, s-butyl, t-butyl, pentyl, hexyl, or octyl group, preferably a C₁₋₆alkyl group, and more preferably a C₁₋₄alkyl group. The aryl group includes, for example, a C₆₋₂₀aryl group such as phenyl, naphthyl, or biphenyl group, preferably a C₆₋₁₈aryl group, and more preferably a C₆₋₁₄aryl group, particularly phenyl group. As the aralkyl group, there may be, for example, a C₇₋₂₀aralkyl group (e.g., a C₆₋₁₂aryl-C₁₋₈alkyl group) such as benzyl or phenethyl group, preferably a C₆₋₁₂aryl-C₁₋₆alkyl group, particularly benzyl group. The alkoxy group may be, for example, a C₁₋₂₀alkoxy group such as methoxy, ethoxy, propoxy, butoxy, or t-butoxy group, preferably a C₁₋₁₀alkoxy group, and more preferably a C₁₋₆alkoxy group.

The substituent R³ practically includes a hydrogen atom, an alkyl group (for example, a linear or branched C₁₋₆alkyl group), a C₆₋₁₂aryl group (e.g., phenyl group), a C₆₋₁₂aryl-C₁₋₄alkyl group, and a linear of branched C₁₋₆alkoxy group. In particular, the substituent R³ is usually a hydrogen atom, or an alkyl group (for example, a linear or branched C₁₋₆alkyl group).

The hydrocarbon ring represented by the ring Z may be a non-aromatic hydrocarbon ring (e.g., a C₃₋₂₀cycloalkane ring such as cyclohexane ring or cyclooctane ring, a C₃₋₂₀cycloalkene ring such as cyclohexene ring), and the ring Z is usually an aromatic hydrocarbon ring. The aromatic hydrocarbon ring may have at least a benzene ring essentially, and includes, for example, benzene ring and a condensed polycyclic aromatic hydrocarbon ring (e.g., naphthalene ring, anthracene ring, phenanthrene ring, phenalene ring). As the preferred hydrocarbon ring, there may be exemplified a C₆₋₂₀hydrocarbon ring such as benzene ring, naphthalene ring, or phenalene ring (in particular a C₆₋₁₀hydrocarbon ring).

The heterocycle represented by the ring Z includes heterocycles having at least one hetero atom selected from nitrogen, oxygen, and sulfur atoms, and these heterocycles may be a condensed heterocycle in which a plurality of heterocycles are condensed each other or a condensed heterocycle in which a heterocycle is condensed (e.g., ortho-condensed, ortho and peri-condensed) with a hydrocarbon ring (a non-aromatic hydrocarbon ring, or an aromatic hydrocarbon ring), as well as monocyclic heterocycles. The heterocycle represented by the ring Z may be non-aromatic, and is practically aromatic.

Examples of the heterocycle having a nitrogen atom as a hetero atom are a 5- or 6-membered monocyclic heterocycle such as pyrrole, imidazole, pyridine, or pyrazine ring; and a condensed heterocycle in which a 5- or 6-membered heterocycle is condensed with a hydrocarbon ring, such as indoline, quinoline, isoquinoline, quinazoline, carbazole, phenanthridine, acridine, or phenazine ring. As the heterocycle having an oxygen atom as a hetero atom, there are exemplified a 5- or 6-membered monocyclic heterocycle such as furan ring, and a condensed heterocycle in which a 5- or 6-membered heterocycle is condensed with a hydrocarbon ring, such as isobenzofuran ring or chromene ring. The examples of the heterocycle having a sulfur atom as a hetero atom include a 5- or 6-membered monocyclic heterocycle such as thiophene ring; and a condensed heterocycle in which a 5- or 6-membered heterocycle is condensed with a hydrocarbon ring, such as thianthrene ring. Exemplified as the heterocycle having different hetero atoms is a 5- or 6-membered monocyclic heterocycle such as morpholine, isothiazole, or isoxazole ring; and a condensed heterocycle in which a 5- or 6-membered heterocycle is condensed with a hydrocarbon ring, such as phenoxathiin ring.

The preferred heterocycle includes an aromatic heterocycle, e.g., a 5- or 6-membered heterocycle having a nitrogen atom as a hetero atom (e.g., pyrrole ring, pyridine ring); and an aromatic heterocycle (e.g., carbazole ring) having an aromatic hydrocarbon ring (particularly, benzene ring or naphthalene ring) condensed with a 5- or 6-membered heterocycle having at least a nitrogen atom as a hetero atom.

Incidentally, the ring Z (aromatic ring) has usually a bonding site on the aromatic ring to form a conjugated system comprising the ring Z and the adjacent C═C bond. Moreover, in the case of a polycyclic ring, insofar as the ring Z has a bonding site on the aromatic ring, it does not matter whether the other ring or rings are non-aromatic or aromatic ones, and a part of the ring Z (or a part of the non-conjugated site) maybe hydrogenated. As the hydrocarbon ring partially hydrogenated, there may be mentioned, for example, a hydrogenated naphthalene ring such as 1,2-dihydronaphthalene ring, a hydrogenated phenalene ring such as 2,3-dihydrophenalene or 2,3,3a,4,5,6-hexahydrophenalene ring. Moreover, as the partially hydrogenated heterocycle, there may be mentioned, for example, julolidine ring and 9-formyljulolidine ring.

The ring Z may have a variety of substituents, and examples of which may be a linear or branched C₁₋₆alkyl group such as methyl, ethyl, butyl, or t-butyl group; a C₃₋₁₀cycloalkyl group such as cyclohexyl group; a C₆₋₁₈aryl group such as phenyl group; a C₆₋₁₂aryl-C₁₋₄alkyl group such as benzyl or diphenylmethyl group; a halogen atom (fluorine atom, chlorine atom, bromine atom, and iodine atom); a hydroxyl group; a linear or branched C₁₋₆alkoxy group such as methoxy group, ethoxy group, butoxy group, or t-butoxy group; a hydroxyC₁₋₆alkyl group such as hydroxymethyl group; a carbonyl group; a carboxyl group; a linear or branched C₁₋₄alkoxy-carbonyl group; a linear or branched C₁₋₆alkyl-carbonyl group; a C₆₋₁₂aryl-carbonyl group; a linear or branched C₁₋₆acyloxy group such as acetyloxy group; a cyano group; an amino group; a N-substituted amino group (e.g., a mono- or diC₁₋₆alkylamino group such as methylamino group, dimethylamino group, ethylamino group, diethylamino group, methylethylamino group, propylamino group, diisopropylamino group, butylamino group, or dibutylamino group, a mono- or diC₆₋₁₈arylamino group such as phenylamino group, a C₁₋₆acylamino group such as acetamide group); a nitro group; and a sulfonyl group (or sulfo group).

The preferred substituent includes, for example, a linear or branched C₁₋₄alkyl group, a C₆₋₁₂aryl group, a hydroxyl group, a linear or branched C₁₋₄alkoxy group, an amino group, a mono- or diC₁₋₆alkylamino group (preferably a mono- or diC₁₋₄alkylamino group, and more preferably a diC₁₋₄alkylamino group), a mono- or diC₆₋₁₈arylamino group, a C₁₋₄acyloxy group, and a C₁₋₄acylamino group. As the substituent(s), an electron donative group (e.g., at least one member selected from the group consisting of an amino group, a N-substituted amino group, a hydroxyl group, an alkoxy group, a halogen atom, an alkyl group, and others) seems to be preferred.

Incidentally, there is no particular limitation as to the position(s) of the substituent(s) on the hydrocarbon ring or the heterocycle. For example, on the benzene ring, the substituent(s) may be attached on the o-, m-, or p-position, and is usually attached on the o- and/or p-position (in particular, the position of the electron donative group as a substituent is usually at least one position selected from the o-position and p-position). Moreover, the hydrocarbon ring and the heterocycle each may have a plurality of substituents, and a plurality of substituents may be the same or different.

The hydrocarbon ring having such substituent(s) includes, for example, a benzene ring having a substituent(s) (e.g., a benzene ring substituted with at least one substituent selected from a halogen atom, a hydroxyl group, a C₁₋₄alkoxy group, an amino group, and a mono- or diC₁₋₄alkyl-substituted amino group). Moreover, the heterocycle having a substituent(s) includes a N-substituted heterocycle in which its hetero atom(s) (e.g., nitrogen atom) is substituted for a C₁₋₆alkyl group [e.g., carbazole ring substituted for a N-C₁₋₁₄alkyl group].

In the compound represented by the formula (I), combinations of substituents are exemplified as follows.

X¹: a cyano group

X²: a cyano group

R¹: a C₁₋₆alkyl group

R²: a hydrogen atom or a C₁₋₆alkyl group

R³: a hydrogen atom or a C₁₋₆alkyl group

Z: an aromatic ring (e.g., a C₆₋₂₀aryl ring such as benzene ring or a condensed hydrocarbon ring, or a condensed heterocycle in which a heterocycle is condensed with an aromatic hydrocarbon ring) having a substituent(s) (at least one substituent selected from an amino group, a N-substituted amino group, a hydroxyl group, an alkoxy group, a halogen atom, and an alkyl group), or a ring in which a part of the unconjugated site is hydrogenated.

The typical compound (I) includes, for example, a compound in which the ring Z is a benzene ring which may have a substituent [for example, a 2,3-dicyano-5alkyl-6-alkyl-7-(2-phenylethen-1-yl)-6H-1,4-diazepine such as 2,3-dicyano-5,6-dimethyl-7-(2-phenylethen-1-yl)-6H-1,4-diazepine, 2,3-dicyano-5-methyl-6-ethyl-7-(2-phenylethen-1-yl)-6H-1,4-diazepine, or 2,3-dicyano-5-methyl-6-butyl-7-(2-phenylethen-1-yl)-6H-1,4-diazepine; a 2,3-dicyano-5-alkyl-6,6-dialkyl-7-(2-phenylethen-1-yl)-6H-1,4-diazepine such as 2,3-dicyano-5,6,6-trimethyl-7-(2-phenylethen-1-yl)-6H-1,4-diazepine, 2,3-dicyano-5-methyl-6,6-diethyl-7-(2-phenylethen-1-yl)-6H-1,4-diazepine, or 2,3-dicyano-5ethyl-6,6-dibutyl-7-(2-phenylethen-1-yl)-6H-1,4-diazepine; a compound in which a 5-positioned alkyl group of the above azepine compound is substituted for a hydrogen atom, a phenyl group, or other group; a compound in which a 7-positioned 2-phenylethen-1-yl group of the above azepine compound is substituted for 2-phenylethen-1-yl group having an electron donative group (such as an amino group, a N-substituted amino group, a hydroxyl group, an alkoxy group, a halogen atom, or an alkyl group) on the 4-position of the phenyl group (e.g., 2-(4-alkoxyphenyl)ethen-1-yl group, 2-(4-mono- or dialkylaminophenyl)ethen-1-yl group); a compound in which a 5-positioned alkyl group of the above compound is substituted for a hydrogen atom, a phenyl group, or other group, and a 7-positioned 2-phenylethen-1-yl group thereof is substituted for 2-phenylethen-1-yl group having an electron donative group (such as an amino group, a N-substituted amino group, a hydroxyl group, an alkoxy group, a halogen atom, or an alkyl group) on the 4-position of the phenyl group], a compound in which the ring Z is a condensed polycyclic hydrocarbon ring, or an aromatic heterocycle in which a heterocycle is condensed with an aromatic hydrocarbon ring, or a partially hydrogenated ring thereof [for example, a 2,3-dicyano-5-alkyl-6-alkyl-7-(2-(phenalen-2-yl)ethen-1-yl)-6H-1,4-diazepine, 2,3-dicyano-5-alkyl-6-alkyl-7-[(9-ethyl-3-carbazolyl)vinyl-1-yl]-6H-1,4-diazepine; a compound in which a 5-positioned alkyl group of the above azepine compound is substituted for a hydrogen atom, a phenyl group, or other group], and others.

### Production Process

The compound of the present invention may for example be prepared by a reaction of a compound represented by the following formula (IV) with a compound represented by the following formula (V). Moreover, the compound represented by the formula (IV) may for example be obtained by a reaction of a compound represented by the following formula (II) with a compound represented by the following formula (III). The reaction scheme using these compounds is shown as follows.

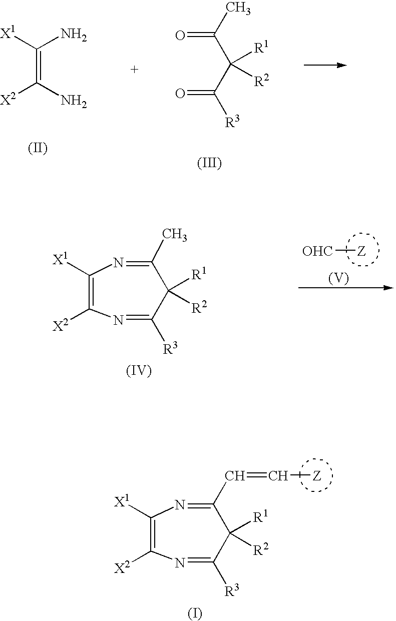

wherein X¹, X², R¹, R², R³ and the ring Z have the same meanings as defined above.

The compound represented by the formula (II) (including a constitutional isomer thereof) may be allowed to react with the compound represented by the formula (III) to give the compound represented by the formula (IV).

The typical compound represented by the formula (II) includes, for example, a diamine compound [e.g., 1,2-dicyano-1,2-diaminoethene (diaminomaleonitrile), 1-cyano-2-(dimethylamino)-1,2-diaminoethene, 1,2-dicyano-2-(benzylamino)-1-aminoethene)]. The typical compound represented by the formula (III) includes, for example, a diketone compound [for example, a 2-alkyl-butane-1,3-dione such as 2-methyl-butane-1,3-dione; a 2,2-dialkyl-butane-1,3-dione such as 2,2-dimethyl-butane-1,3-dione; a 3-alkyl-pentane-2,4-dione such as 3-methyl-pentane-2,4-dione, a 3,3-dialkyl-pentane-2,4-dione such as 3,3-dimethyl-pentane-2,4-dione; a 3-alkyl-hexane-2,4-dione such as 3-methyl-hexane-2,4-dione, a 3,3-dialkyl-hexane-2,4-dione such as 3,3-dimethyl-hexane-2,4-dione; a 3-alkyl-C₇₋₂₀alkane-2,4-dione, a 3,3-dialkyl-C₇₋₂₀alkane-2,4-dione, a 1-phenyl-2-alkyl-butane-1,3-dione, a 1-aralkyl-2-alkyl-butane-1,3-dione, a 1-alkoxy-2-alkyl-butane-1,3-dione]. Incidentally, such diamine and diketone compounds may be commercial products or may be produced by conventional manners.

The amount of the compound (III) is usually about 1 to 3 mol, and preferably about 1 to 1.5 mol relative to 1 mol of the compound (II).

The reaction (condensation reaction) described above may be conducted in the presence or absence of a catalyst. Exemplified as the catalyst are conventional ones, such as an acid catalyst (e.g., an inorganic acid such as sulfuric acid, phosphoric acid, or hydrochloric acid; an organic acid such as acetic acid, oxalic acid, sulfonic acid, or p-toluenesulfonic acid) and a basic catalyst (e.g., an amine such as piperidine, a hydroxide or oxide of an alkali metal or alkaline earth metal). The amount of the catalyst may be selected within the range of about 0.001 to 1 mol, relative to 1 mol of the compound (II). Moreover, the reaction may be carried out in the presence of a dehydrating agent (e.g., phosphorus pentoxide).

The condensation reaction may be conducted in a solvent inert to the reaction. As the solvent, there may for example be mentioned an aliphatic hydrocarbon (e.g., hexane), an alicyclic hydrocarbon (e.g., cyclohexane), an aromatic hydrocarbon (e.g., benzene, toluene), a halogenated hydrocarbon (e.g., chloroform), an alcohol (e.g., methanol, ethanol, isopropyl alcohol, butanol), an ester (e.g., ethyl acetate, butyl acetate, isobutyl acetate), an ether (e.g., dioxane, diethyl ether, teterahydrofuran), an amide (e.g., formamide, acetamide, dimethylformamide (DMF), dimethylacetamide), a nitrile (e.g., acetonitrile, benzonitrile), a sulfoxide (e.g., dimethyl sulfoxide), and others. Moreover, when the solvent is used, the reaction temperature may be selected within the range of about 0° C. to a reflux temperature, and is for example about 50 to 120° C. and preferably about 60 to 100° C. It is possible to conduct the reaction under ordinary, reduced, or applied pressure. The reaction may be conducted in an atmosphere of an inert gas (e.g., nitrogen, argon, helium). After the completion of the reaction, the compound formed by the condensation reaction described above may be easily separated and purified by such a conventional means as filtration, condensation, distillation, extraction, crystallization, recrystallization, column chromatography, or a combination means thereof.

The compound (I) of the present invention may be obtained by allowing a compound (IV) produced by the reaction to react with the aldehyde compound (V).

The typical compound (IV) includes, for example, a 2,3-dicyanodiazepine corresponding to the above-mentioned compound (I), for example, 2,3-dicyano-5-alkyl-6-alkyl-7-methyl-1,4-diazepine, 2,3-dicyano-5-alkyl-6,6-dialkyl-7-methyl-1,4-diazepine, or others.

The typical compound (V) includes, for example, an aldehyde compound corresponding to the above-mentioned formula (V) [e.g., an aldehyde in which the ring z is a benzene ring (e.g., benzaldehyde, a halobenzaldehyde, aminobenzaldehyde, a N-substituted aminobenzaldehyde (particularly, a N-C₁₋₄alkyl substituted aminobenzaldehyde), phenol-aldehyde, a C₁₋₄alkoxybenzaldehyde); an aldehyde in which the ring Z is a condensed polycyclic hydrocarbon ring (e.g., naphthalenecarbaldehyde, phenalenecarbaldehyde); an aldehyde in which the ring Z is a 5- or 6-membered heterocycle containing a nitrogen atom(s) as a hetero atom, or a condensed heterocycle of a heterocycle and a hydrocarbon ring (e.g., 9-ethyl-3-formylcarbazole)].

The amount of the compound (V) is about 1 to 3 mol, and preferably about 1 to 1.5 mol relative to 1 mol of the compound (IV).

The reaction may be carried out in the presence of a solvent inert to the reaction, such as a solvent exemplified above. If necessary, a catalyst (e.g., a basic catalyst such as pyridine or piperidine) may be used. The amount of the catalyst may be selected within the range of, relative to 1 mol of the compound (IV), about 0.001 to 1 mol.

In the case using a solvent, the reaction temperature may be selected within the range of about 0° C. to a reflux temperature, and is for example about 50 to 120° C. and preferably about 60 to 100° C. The reaction may be conducted under an ordinary, reduced, or applied pressure. The reaction may be carried out in an atmosphere of an inert gas (e.g., nitrogen, argon, helium).

After the completion of the reaction, the compound (I) formed in the above-described reaction may be easily separated and purified by such a conventional means as filtration, condensation, distillation, extraction, crystallization, recrystallization, column chromatography, or a combination means thereof.

In the case where the ring Z is an aromatic ring, the compound (I) forms a conjugated system between the azepine ring and the ring Z via a carbon-carbon double bond by the reaction of (IV) with (V).

The compound of the present invention is characterized in that, due to its specific structure, it is capable of emitting light by supply of an external energy (irradiation of light, the action of an electric field). There is no particular limitation as to the light irradiation so far as the irradiated light has a certain wavelength capable of exciting the azepine compound (I). For example, ultraviolet rays (not longer than 400 nm) and visible rays [about 360 to 860 nm (preferably about 400 to 760 nm, and more preferably about 400 to 700 nm)] can be used. The emission wavelength may vary depending on, for example, the species of the substituent(s) and the substitution position, and is usually in a region of relatively longer wavelength [e.g., a wavelength of about 450 to 750 nm, preferably about 500 to 700 nm (red light), in particular about 530 to 700 nm (yellow to red light)]. Further, the azepine compound (I) of the present invention has a large molar absorption coefficient which varies for different species of substituents or positions of substitution.

Under the action of an electric field (or applying of a voltage, injection of a carrier), the compound of the present invention emits light (fluorescence). In particular, the compound (I) of the present invention ensures high luminance or light intensity. The emission wavelength is selectable within the above-mentioned wavelength range, and the compound (I) is capable of emitting light even in a relatively longer wavelength region [about 500 to 700 nm, and preferably about 550 to 700 nm (yellow to red light)]. Moreover, surprisingly, introduction of an alkyl group (such as ethyl group or butyl group) as a substituent of at least one of the groups, R¹ and R², into the azepine ring achieves red shift of wavelength (emission wavelength) (or shift to longer wavelength) compared with a compound in which both of R¹ and R² are a hydrogen atom, as apparent from FIG. 2 described below. Thus, it is expected that the alkyl group-introduced compound provides a brilliant red luminescence, and such a compound is a potential extremely useful pigment. Therefore, the compound of the present invention is useful for an emission center compound of an organic EL device. Moreover, the present invention also includes a method comprising introducing an alkyl group as a substituent of at least one of the groups, R¹ and R², into the azepine ring to cause red shift of the emission wavelength (or shift to longer wavelength).

Further, the compound (I) in the solid form (e.g., a vapor-deposited thin film) shows the same properties (light emission upon light irradiation or by the action of an electric field) as well as in the form of a solution. Therefore, the compound of the present invention can be used in the form of not only a liquid form but also a solid form (e.g., a thin film or film, powder, particles), and its application is not restricted.

Since the compound of the present invention is capable of emitting light by light irradiation or an electric field application, it can be utilized in various fields as a functional material. For example, the compound of the present invention is useful not only as a fluorescent material (e.g., a fluorescent pigment, a fluorescent flaw detecting agent, a fluorescent dye such as a fluorescent white dye, particularly a fluorescent material such as a fluorescent dye) but also as a material for display (e.g., a material for a light emitting device such as an electroluminescent material).

### Organic Electroluminescent Device

The electroluminescent (EL) device of the present invention is composed of a pair of electrodes and an organic layer interposed therebetween. The organic layer comprises at least the compound represented by the aforementioned formula (I). In particular, a layer containing the compound (I) forms a light-emitting region, constituting a light-emitting layer. The light-emitting layer may be formed with a film-formable compound of the formula (I) alone, or may be formed with a film-formable or non-film-formable compound (I) and a binder having a film-forming (or film-formable) property. As the binder, a resin having a film-forming (or film-formable) property (a thermoplastic resin, a thermosetting resin) may be usually used.

Examples of the thermoplastic resin includes an olefinic resin such as a polyethylene, a polypropylene, an ethylene-propylene copolymer, or a polybutene; a styrenic resin such as a polystyrene, a rubber-modified or rubber-grafted polystyrene (e.g., HIPS), an acrylonitrile-styrene copolymer, or an acrylonitrile-butadiene-styrene copolymer; an acrylic resin [e.g., a homo- or copolymer of a (meth)acrylic monomer (e.g., a C₁₋₆alkyl (meth)acrylate such as methyl (meth)acrylate, ethyl (meth)acrylate, or butyl (meth)acrylate; a hydroxyC₂₋₄alkyl (meth)acrylate such as hydroxyethyl (meth)acrylate or hydroxypropyl (meth)acrylate; glycidyl (meth)acrylate; (meth)acrylic acid; (meth)acrylonitrile); a copolymer of the (meth)acrylic monomer mentioned above with a copolymerizable monomer (e.g., an aromatic vinyl monomer such as styrene) (e.g., a methyl methacrylate-styrene copolymer)]; a vinyl-series resin such as a vinyl alcohol-series polymer such as a polyvinyl alcohol and an ethylene-vinyl alcohol copolymer, a polyvinyl chloride, a vinyl chloride-vinyl acetate copolymer, a polyvinylidene chloride, a polyvinyl acetate, or an ethylene-vinyl acetate copolymer; a polyamide-series resin such as a 6-nylon, a 6,6-nylon, a 6,10-nylon, or a 6,12-nylon; a polyester resin [e.g., an alkylene arylate-series resin or alkylene arylate copolyester resin such as a polyalkylene terephthalate (e.g., a polyethylene terephthalate, a polybutylene terephthalate) or a polyalkylene naphthalate]; a fluorine-containing resin; a polycarbonate; a polyacetal; a polyphenylene ether; a polyphenylene sulfide; a polyether sulfone; a polyether ketone; a thermoplastic polyimide; a thermoplastic polyurethane; and a norbornene-series polymer.

The thermosetting resin includes a phenolic resin, an amino resin (e.g., a urea resin, a melamine resin), a thermosetting acrylic resin, an unsaturated polyester resin, an alkyd resin, a diallyl phthalate resin, an epoxy resin, and a silicone resin.

These binders may be used either singly or in combination.

The proportion of the compound (I) is not particularly limited as far as the film-formability would not be deteriorated, and may for example be about 0.01 to 25 parts by weight, preferably about 0.05 to 10 parts by weight, more preferably about 0.1 to 5 parts by weight relative to 100 parts by weight of the binder.

If necessary, into the light-emitting layer may be incorporated other emission center compounds, examples of which are a heterocyclic compound having at least one hetero atom selected from oxygen, nitrogen, and sulfur atoms [e.g., a bis(C₁₋₆alkyl-benzoxazoyl)thiophene typified by 2,5-bis(5-tert-butyl-2-benzoxazoyl)-thiophene; nile red; a coumarin such as coumarin 6 and coumarin 7; a 4-(dicyanoC₁₋₄alkylene)-2-C₁₋₄alkyl-6-(p-diC₁₋₄alkylaminostyryl)-4H-pyran typified by 4-(dicyanomethylene)-2-methyl-6-(p-dimethylaminostyryl)-4H-pyran; and quinacridone]; a condensed polycyclic hydrocarbon such as rubrene or perylene; a tetraC₆₋₁₂aryl-1,3-butadiene such as 1,1,4,4-tetraphenyl-1,3-butadiene (TPB); a bis(2-(4-C₁₋₄alkylphenyl)C₂₋₄alkynyl)benzene such as 1,4-bis(2-(4-ethylphenyl)ethynyl)benzene; and a bis(2,2′-diC₆₋₁₂arylvinyl)biphenyl such as 4,4′-bis(2,2′-diphenylvinyl)biphenyl. These emission center compounds may be used either singly or in combination. The content of the emission center compound is selected within a range not adversely affecting the emission efficiency of the compound (I) and may be about 0.01 to 10 parts by weight, about 0.05 to 5 parts by weight, and more preferably about 0.1 to 3 parts by weight relative to 100 parts by weight of the binder. The proportion of the compound (I) relative to the other emission center compound(s) [the former/the latter (weight ratio)] may be about 40/60 to 100/0, preferably about 50/50 to 95/5, and more preferably about 60/40 to 90/10.

If necessary, the light-emitting layer comprising the compound (I) may be given an electron-transportability (or electron-transporting function) and/or a hole-transportability (or hole-transporting function). For the purpose of giving such a function(s), (1) to the light-emitting layer may be added organic polymers or compounds having the functions described above; or (2) the light-emitting layer may be laminated with a layer or layers having the functions described above. In the embodiment (1), it is possible to form an organic EL device having a single-layered structure.

The organic polymer having at least one function selected from the electron-transportability and hole-transportability includes, for example, a vinyl-series polymer having at least one functional group selected from hole-transporting functional groups and electron-transporting functional groups in the main chain or side chain, such as a polyphenylenevinylene in which the vinylene group is inserted between the phenylene groups [e.g., a homo- or copolymer of a C₆₋₁₂arylenevinylene which may have a substituent (e.g., a C₁₋₁₀alkoxy group), such as a polyphenylenevinylene, a poly(2,5-dimethoxyphenylenevinylene, or a polynaphthalenevinylene]; a polyphenylene (particularly, a polyparaphenylene) [e.g., a homo- or copolymer of a phenylene which may have a substituent (e.g., a C₁₋₁₀alkoxy group), such as a polyparaphenylene or a poly-2,5-dimethoxyparaphenylene]; a polythiophene [e.g., a polyC₁₋₂₀alkylthiophene such as a poly(3-alkylthiophene); a polyC₃₋₂₀cycloalkylthiophene such as a poly(3-cyclohexylthiophene); a homo- or copolymer of a C₆₋₂₀arylthiophene which may have a substituent (e.g., a C₁₋₁₀alkyl group) such as a poly(3-(4-n-hexylphenyl)thiophene)]; a polyfluorene such as a polyC₁₋₂₀alkylfluorene; a vinyl-series polymer having at least one functional group selected from a hole-transporting functional group and an electron-transporting functional group in the main or side chain, such as a poly-N-vinylcarbazole (PVK), a poly-4-N,N-diphenylaminostyrene, a poly(N-(p-diphenylamino)phenylmethacrylamide), a poly(N,N′-diphenyl-N,N′-bis(3-methylphenyl)-1,1′-biphenyl-4,4′-diaminomethacrylamide) (PTPDMA), or a poly-4-(5-naphthyl-1,3,4-oxadiazole)styrene; a polyC₁₋₄alkylphenylsilane such as a polymethylphenylsilane; a polymer having an aromatic amine derivative in the side chain or main chain; a copolymer of these polymers; and others. These resins may be used either singly or in combination. The preferred resin includes a poly-N-vinylcarbazole or a copolymer containing N-vinylcarbazole as amain component (not less than 50% by weight, preferably about 60 to 98% by weight), and a polymer having an aromatic amine derivative in the main or side chain.

PVK is amorphous and excellent in heat resistance (glass transition temperature Tg: 224° C.). There is no particular restriction on the degree of polymerization of PVK, and may for example be about 100 to 1,000, and preferably about 200 to 800.

In the case where the light-emitting layer comprises the compound (I) and the organic polymer described above, the content of the compound (I) may be about 0.01 to 10 parts by weight, preferably about 0.05 to 5 parts by weight, and more preferably about 0.1 to 3 parts by weight relative to 100 parts by weight of the organic polymer.

If necessary, to the light-emitting layer comprising the compound of the formula (I) and the organic polymer may be added a compound having an electron-transportability or hole-transportability.

The compound having an electron-transportability includes, for example, an oxadiazole derivative [e.g., an oxadiazole derivative having a C₆₋₂₀aryl group which may have a substituent, such as 2-(4-biphenyl)-5-(4-tert-butylphenyl)-1,3,4-oxadiazole (PBD), 2,5-bis(1-naphthyl)-1,3,4-oxadiazole (BND), 1,3-bis[5-(4-tert-butylphenyl)-1,3,4,-oxadiazole)]benzene (BPOB), 1,3,5-tris[5-(4-tert-butylphenyl)-1,3,4-oxadiazole]benzene (TPOB), or 1,3,5-tris[5-(1-naphthyl)-1,3,4-oxadiazole]benzene (TNOB); a diphenoquinone [e.g., a diphenoquinone which may have a substituent (e.g., a C₁₋₁₀ alkyl group), such as 3,5,3′,5′-tetrakis-tert-butyldiphenoquione]; 1,2,3,4,5-pentaphenyl-1,3-cyclopentadiene (PPCP); and a quinolinolato complex such as a tris(8-quinolinolato)aluminum (III) complex, a bis(benzoquinolinolato)beryllium complex, or a tris(10-hydroxybenzo[h]quinolinolato)beryllium complex. PBD is particularly preferred one.

As the compound having a hole-transportability, there may be exemplified an aromatic tertiary amine such as N,N′-diphenyl-N,N′-bis(3-methylphenyl)-1,1′-biphenyl-4,4′-diamine (TPD), N,N′-diphenyl-N,N′-bis(1-naphthyl)-1,1′-biphenyl-4,4′-diamine (NPD), 1,1bis[(di-4-tolylamino)phenyl]cyclohexane, N,N,N′N′-tetra(3-methylphenyl)-1,3-diaminobenzene (PDA), 4,4′,4″-tris(3-methylphenylphenylamino)triphenylamine (m-MTDATA), 4,4′,4″-tris(1-naphthylphenylamino)triphenylamine(1-TNATA), 4,4′,4″-tris(1-naphthylphenylamino)triphenylamine (1-TNATA), 4,4′,4″-tri(N-carbazolyl)triphenylamine (TCTA), 1,3,5-tris[4-(3-methylphenylphenylamino)phenyl]benzene (m-MTDAPB), or triphenylamine; and a phthalocyanine.

The compounds having an electron-transportability or a hole-transportability may be used either singly or in combination. The content of the compound having an electron and/or hole-transportability may be, relative to 100 parts by weight of the binder (and/or the organic polymer described above), about 10 to 200 parts by weight, preferably about 30 to 150 parts by weight, and more preferably about 50 to 130 parts by weight.

Incidentally, in the case where the light-emitting layer is lacking in either the electron-transportability or the hole-transportability, or enhances each function, a layer or layers having the desired function may be applied onto the light-emitting layer by a conventional method (e.g., vapor deposition, solution coating). These layers may comprise low molecular weight compounds or high molecular weight compounds.

The thickness of each layer constituting the organic layer is not particularly limited, and is for example about 1 nm to 1 μm, preferably about 5 to 800 nm, more preferably about 10 to 500 nm, and particularly about 15 to 300 nm.

As the anode of the organic EL device, for example, a transparent electrode (e.g., an electrode composed of tin oxide, indium-tin-oxide (ITO), or the like) formed by a conventional process (e.g., vacuum deposition) may be employed. As the cathode, a highly conductive metal of low work function (e.g., magnesium, lithium, aluminum, silver) is practically used. In the case where magnesium is employed as the cathode, for improving the adhesion to a film of organic EL devices, magnesium may be co-deposited together with a small amount of silver (e.g., 1 to 10% by weight).

There is no particular restriction on the process for producing the organic electroluminescent device of the present invention, and conventional ones may be utilized. For example, the organic layer (e.g., light-emitting layer) may be formed by forming a thin layer of the aforementioned transparent electrode (e.g., ITO electrode) on a transparent substrate and then applying or casting a coating solution containing the compound of the formula (I) on the transparent electrode in a conventional manner (e.g., spin coating, casting). The organic electroluminescent device is produced by further forming a cathode on the organic layer by vapor deposition or other means. If necessary, the anode or the light-emitting layer may be laminated with a layer or layers having an electron- and/or hole-transportability by such a conventional method as vapor deposition or coating.

Examples of the substrate are those transparent enough to transmit light emitted by the emission center compound, such as glass plates (e.g., a soda glass, a non-alkali glass, and a quartz glass), sheets or films of polymers (e.g., a polyester, a polysulfone, and a polyethersulfone). For producing a flexible organic EL device, a polymer film is preferably used.

The total thickness of the organic EL device (e.g., the organic layer and the electrodes) as a whole may not be particularly limited, and may be about 50 nm to 10 μm, preferably about 100 nm to 8 μm, and more preferably about 300 nm to 5 μm.

According to the present invention, since a specific azepine compound having a non-planer structural azepine ring site is employed as the organic layer (particularly, light-emitting layer) for an element of the organic EL device, luminescence having relatively longer wavelength (e.g., about 530 to 700 nm) can be emitted with high luminance without causing concentration quenching and an organic EL device excellent in durability can be obtained.

The compound of the present invention can emit light by being light-irradiated or by the action of an electric field because it has a specific azepine ring and a specific ring Z. In particular, the compound can emit light of a long wavelength region (for example, emission in a red light region) at high emission luminance. Therefore, the compound of the present invention is useful for a functional material such as a fluorescent material and a material for display devices. In particular, the compound of the present invention is useful for an emission center compound applicable to an organic EL device.

### EXAMPLES

The following examples are intended to describe this invention in further detail and should by no means be interpreted as defining the scope of the invention.

### Synthesis Example 1

(Step 1)

To a benzene solution (4 ml) containing 1 mmol (108 mg) of diaminomaleonitrile were added 0.1 mmol (9 mg) of oxalic acid and 1 mmol of pentane-2,4-dione, and the mixture was subjected to reflux for 5 hours. After removing the solvent from the reaction mixture, the resultant product was isolated by column chromatography on silica gel (eluate: chloroform), and purified by recrystallization from benzene to give 2,3-dicyano-6H-1,4-diazepine (compound IVa) in 78% yield.

Melting point: 189 to 190° C.

¹H NMR (CDCl₃) δ: 1.85; (broad, s, 1H), 2.30; (s, 6H), 4.27; (broad, s, 1H).

EIMS (70 eV) m/z (relative intensity): 172(M⁺)

| Elemental analysis | C (%) | H (%) | N (%) |
|---|---|---|---|
| Calculated | 62.78 | 4.68 | 32.54 |
| Found | 63.00 | 4.61 | 32.04 |

(Step 2)

To a benzene solution (15 ml) containing the obtained compound IVa (1 mmol) were added 1 mmol (177 mg) of 4-diethylaminobenzaldehyde and five drops of piperidine. The mixture was subjected to reflux for 6 hours in a flask provided with Dean-Stark trap to remove generated water therefrom. From the reaction mixture, the solvent was further removed and the resultant product was isolated by column chromatography on silica gel (eluate: chloroform/ethyl acetate=9/1), and purified by recrystallization from benzene to give 2,3-dicyano-5-[4-(diethylamino)styryl]-6H-1,4-diazepine (compound Ia).

Melting point: not less than 300° C.

¹H-NMR(CDCl₃) δ: 1.21 (t, J=7.2 Hz, 6H), 1.59 (s, 3H), 1.83 (broad, s, 1H), 3.43 (q, J=7.2Hz, 4H), 4.57 (broad, s, 1H), 6.67 (d, J=15.9 Hz, 1H), 6.68 (d, J=8.7 Hz, 2H), 7.44 (d, J=15.9 Hz, 1H), 7.45 (d, J=8.7 Hz, 2H)

EIMS (70 eV) m/z (relative intensity): 331(M⁺)

| Elemental analysis | C (%) | H (%) | N (%) |
|---|---|---|---|
| Calculated | 72.48 | 6.39 | 21.13 |
| Found | 72.63 | 6.40 | 20.41 |

### Synthesis Example 2

(Step 1)

With the exception that 3-ethyl-pentane-2,4-dione was used as a diketone compound, 2,3-dicyano-6-ethyl-6H-1,4-diazepine (compound IVb) was obtained in the same manner as in the step 1 of Synthesis Example 1. Incidentally, ethyl acetate was used as an eluate for column chromatography.

Melting point: 180 to 182° C.

¹H-NMR (CDCl₃) δ: 1.51 (t, J=7.4 Hz, 3H), 1.27 (t, J=7.4 Hz, 1H), 2.15 (s, 6H), 2.21-2.36 (m, 2H)

EIMS (70 eV) m/z (relative intensity): 200 (M⁺; 77), 185 (100)

| Elemental analysis | C (%) | H (%) | N (%) |
|---|---|---|---|
| Calculated | 65.98 | 6.04 | 27.98 |
| Found | 66.00 | 6.05 | 27.97 |

(Step 2)

The object compound Ib, 2,3-dicyano-5-[4-(diethylamino)styryl]-6-ethyl-6H-1,4-diazepine was obtained in the same manner as in the step 2 of Synthesis Example 1 with the exception that the obtained compound IVb was used in lieu of the compound IVa. Incidentally, chloroform/ethyl acetate=10/1 was used as an eluate for column chromatography, and the compound Ib was purified by recrystallization from toluene.

Melting point: 196 to 198° C.

¹H-NMR (CDCl₃) δ: 1.17 (t, J=7.4 Hz, 3H), 1.21 (t, J=7.1 Hz, 6H), 1.39 (t, J=7.4 Hz, 1H), 2.05 (s, 3H), 2.31-2.42 (m, 2H), 3.43 (q, J=7.1 Hz, 4H), 6.31 (d, J=15.0 Hz, 1H), 6.64 (d, J=8.8 Hz, 2H), 7.41 (d, J=8.8 Hz, 2H), 7.68 (d, J=15.0 Hz, 1H)

EIMS (70 eV) m/z (relative intensity): 359 (M³⁰ ; 81), 344 (100)

$$\begin{matrix}Elemental analysis & C ( \% ) & H ( \% ) & N ( \% ) \\ Calculated & 73.51 & 7.01 & 19.48 \\ Found & 73.55 & 7.05 & 19.50\end{matrix}$$

### Synthesis Example 3

(Step 1)

The compound IVc, 2,3-dicyano-6-butyl-6H-1,4-diazepine was obtained in the same manner as in the step 1 of Synthesis Example 1 except for using 3-butyl-pentane-2,4-dione as a diketone compound. Incidentally, ethyl acetate was used as an eluate for column chromatography.

Melting point (decomposition): 122 to 124° C.

¹H NMR(CDCl₃) δ: 0.99 (t, J=7.0 Hz, 3H), 1.32 (t, J=7.5 Hz, 1H), 1.42-1.46 (m, 4H), 2.13 (s, 6H), 2.22 (q, J=7.5 Hz, 2H)

EIMS (70 eV) m/z (relative intensity): 228 (M⁺; 51), 186 (44), 185 (83), 172 (52), 171 (50), 55 (100)

| Elemental analysis | C (%) | H (%) | N (%) |
|---|---|---|---|
| Calculated | 68.39 | 7.06 | 24.54 |
| Found | 68.41 | 7.10 | 24.55 |

(Step 2)

The object compound Ic, 2,3-dicyano-5-[4-(diethylamino)styryl]-6-butyl-6H-1,4-diazepine was obtained in the same manner as in the step 2 of Synthesis Example 1 with the exception that the obtained compound IVc was used in lieu of the compound IVa. Incidentally, chloroform/ethyl acetate=10/1 was used as an eluate for column chromatography, and the compound Ic was purified by recrystallization from cyclohexane.

Melting point: 132 to 134° C.

¹H-NMR(CDCl₃) δ: 1.00 (t, J=7.0 Hz, 3H), 1.21 (t, J=7.0 Hz, 6H), 1.27 (broad, s, 1H), 1.42-1.48 (m, 4H), 2.04 (s, 3H), 2.21-2.33 (m, 2H), 3.42. (q, J=7.0 Hz, 4H), 6.30 (d, J=15.0 Hz, 1H), 6.64 (d, J=8.8 Hz, 2H), 7.42 (d, J=8.8 Hz, 2H), 7.67 (d, J=15.0 Hz, 1H)

EIMS (70 eV) m/z (relative intensity): 387 (M⁺; 52), 372 (100)

| Elemental analysis | C (%) | H (%) | N (%) |
|---|---|---|---|
| Calculated | 74.38 | 7.54 | 18.07 |
| Found | 74.40 | 7.53 | 18.06 |

### Example 1

Fifty (50) mg of a poly-N-vinylcarbazole (PVK: manufactured by Kanto Kagaku, K.K.), 50 mg of 2-(4-biphenyl)-5-(4-tert-butylphenyl)-1,3,4-oxadiazole (PBD: manufactured by Aldrich Chemical Company, Inc.), and 0.392 mg of the dicyanoazepine compound Ib obtained in Synthesis Example 2 (R²=ethyl group) were dissolved in 3 ml of toluene to prepare a coating solution. A indium-tin-oxide (ITO) layer was formed on a glass substrate, the coating solution was applied on the ITO layer by spin coating to form an organic coating layer having a thickness of 75 nm (measured using “SURFCOM575A” manufactured by Tokyo Seimitsu Co., Ltd.). On the organic coating layer, an Al/Li electrode 200 nm thick was formed by vacuum deposition of a metal base (manufactured by Kojundo Kagaku, K.K., Li content of 0.78% by weight) to give an organic electroluminescent device.

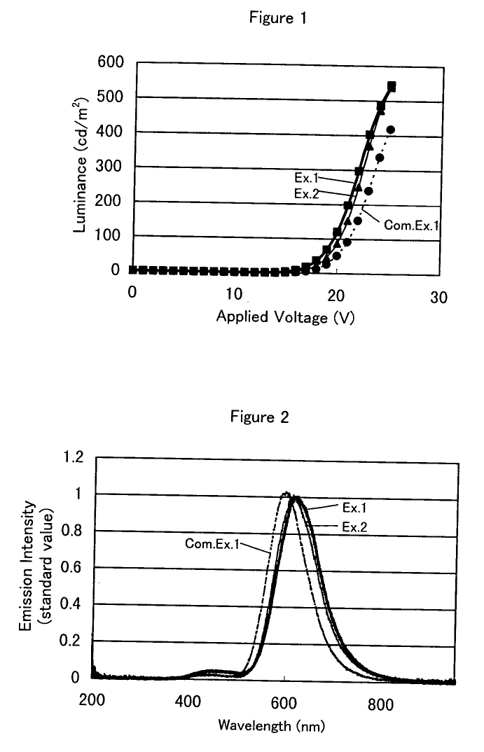

In the obtained organic EL device, the ITO electrode of the organic EL device and the AL/Li electrode were treated as anode and cathode, respectively, a direct electric field was applied between the both electrodes in the atmosphere thereby to make the device emit light. The peak wavelength of the emission spectrum (measured by “Multichannel analyzer PMA-11” manufactured by Hamamatsu Photonics, K.K.) was 623.7 nm. The emission luminance (measured by “Luminance meter LS-110” manufactured by Minolta Co., Ltd.) was 297.8 cd/m² at an applied voltage of 22 V. A graph showing the value of the emission luminance versus the applied voltage is shown in FIG. 1.

### Example 2

The organic EL device was produced in the same manner as in the Example 1 except for using 0.423 mg of the dicyanoazepine compound Ic prepared in Synthesis Example 3 (R²=butyl group) in lieu of 0.392 mg of the dicyanoazepine compound Ib, and the emission spectrum and emission luminance were measured. The thickness of the obtained organic EL device was 75 nm, and the peak wavelength of the emission spectrum thereof was 620.8 nm. Moreover, the emission luminance was 585.7 cd/m at an applied voltage of 26 V.

### Comparative Example 1

The organic EL device was produced in the same manner as in the Example 1 except for using 0.362 mg of the dicyanoazepine compound Ia prepared in Synthesis Example 1 (R²=hydrogen atom) in lieu of 0.392 mg of the dicyanoazepine compound Ib, and the emission spectrum and emission luminance were measured. The thickness of the obtained organic EL device was 75 nm, and the peak wavelength of the emission spectrum thereof was 602.2 nm. Moreover, the emission luminance was 154.4 cd/m² at an applied voltage of 22 V.

In organic EL devices obtained in Examples and Comparative Example, a graph of the value of the emission luminance versus the applied voltage are shown in FIG. 1, and the emission spectra (luminescence intensity distribution) are shown in FIG. 2.

What is claimed is:

1. An azepine compound represented by the following formula (I): 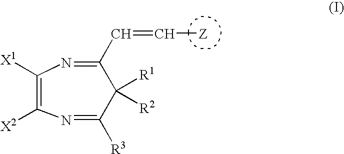
wherein X¹ and X² are the same or different, each representing an electron attractive group; R¹ and R² are the same or different, each representing a hydrogen atom, or an alkyl group, and at least one of the groups, R¹ and R², is an alkyl group; R³ represents a hydrogen atom, an alkyl group, an aryl group, an aralkyl group, or an alkoxy group; and the ring Z represents a hydrocarbon ring which may have a substituent or a heterocycle which may have a substituent.

2. An azepine compound according to claim 1, wherein at least one of the groups, X¹ and X², is a cyano group.

3. An azepine compound according to claim 1, wherein R¹ is a C₁₋₆alkyl group, and R² is a hydrogen atom or a C₁₋₆alkyl group.

4. An azepine compound according to claim 1, wherein R³ is a hydrogen atom or a C₁₋₆alkyl group.

5. An azepine compound according to claim 1, wherein the ring Z is an aromatic ring.

6. An azepine compound according to claim 1, wherein the ring Z is a benzene ring which has an electron donative group selected from the group consisting of an amino group, a N-substituted amino group, a hydroxyl group, an alkoxy group, a halogen atom and an alkyl group, on at least one of the positions, opposition and p-position.

7. An azepine compound according to claim 1, which is capable of emitting light by applying a light or an electric field.

8. A process for producing an azepine compound represented by the following formula (I): 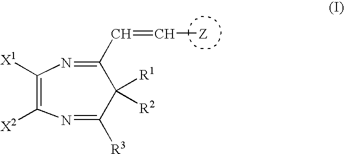
wherein X¹ and X² are the same or different, each representing an electron attractive group; R¹ and R² are the same or different, each representing a hydrogen atom, or an alkyl group, and at least one of the groups, R¹ and R², is an alkyl group; R³ represents a hydrogen atom, an alkyl group, an aryl group, an aralkyl group, or an alkoxy group; and a ring Z represents a hydrocarbon ring which may have a substituent or a heterocycle which may have a substituent,
which comprises reacting a compound represented by the following formula (IV): 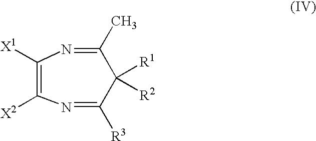
wherein X¹, X², R¹, R², and R³ have the same meanings as defined above,
with a compound represented by the following formula (V): 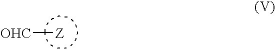
wherein the ring Z has the same meaning as defined above.

9. An organic electroluminescent device, which comprises a pair of electrodes and an organic layer interposed therebetween, wherein the organic layer comprises a compound represented by the formula (I) recited in claim 1.

10. An organic electroluminescent device according to claim 9, wherein the organic layer comprises a light-emitting layer comprising a compound represented by the formula (I).

11. An organic electroluminescent device according to claim 9, wherein the organic layer has (1) a single layer structure composed of a light-emitting layer having at least one function selected from the group consisting of an electron-transportability and a hole-transportability, or (2) a layered structure composed of a layer having at least one function selected from the group consisting of an electron-transportability and a hole-transportability, and a light-emitting layer.
